(19) United States
(12) Patent Application Publication
(10) Pub. No.: US 2010/0028902 A1
(43) Pub. Date: Feb. 4, 2010

(54) LIVING CELL FORCE SENSORS AND METHODS OF USING SAME

(76) Inventors: Scott C. Brown, Gainesville, FL (US); Brij M. Moudgil, Gainesville, FL (US)

Correspondence Address: Beusse Wolter Sanks Mora & Marie, 309 North Orange Avenue, Suite 2500, Orlando, FL, 32801, US

(21) Appl. No.: 12/449,048

(22) PCT Filed: Feb. 26, 2008
(86) PCT No.: PCT/US2008/055044

§ 371 Date: Aug. 24, 2009

Related U.S. Application Data
(60) Provisional application No. 60/891,607, filed on Feb. 26, 2007.

(51) Int. Cl. G01N 33/53; C12M 1/00; C12N 5/00

(52) U.S. Cl. 435/7.1; 435/287.9; 435/395

(57) ABSTRACT

Disclosed herein are materials and methods for the efficient and universal fabrication of microcantilevers terminated with living cells. Methods disclosed describe the passive attachment of cells to microcantilevers that represent cells in suspension comprising living cells attached thereto via association with a hydrophobic layer. Also, disclosed are efficient methods for seeding single and multiple cells to cantilevers that represent isolated adherent cells and tissue constructs of tunable confluency.

### CROSS-REFERENCE TO RELATED APPLICATIONS

This application is related to U.S. Provisional Patent Application No. 60/891,607, filed Feb. 26, 2007, to which priority is claimed under 35 USC 119.

The research which forms the basis of this patent disclosure was supported in part by National Science Foundation Grant No. BES-0609311. Accordingly, the U.S. Government has certain rights in this invention.

### FIELD OF THE INVENTION

The present invention relates to the fabrication of microcantilever-based devices terminated with living cells for the purpose of measuring cell adhesion, cell tribology and other cell-surface interactions.

### GENERAL BACKGROUND

One of the most compelling and difficult problems in modern day science, is the acquisition of an intricate understanding the processes that occur at the cell surface interface. Cell surface interactions are involved in nearly all cell signaling pathways and most physiological cell functions (including survival, proliferation, differentiation, migration or activation, as well as pathological situations such as metastasis formation, tissue invasion by pathogens, atherosclerosis, inflammation, or host-biomaterial interaction). The technological and biomedical significance of cell-surface interactions are, accordingly, large and far-reaching; it is believed that advances in understanding processes that occur at the cell surface will lead to the breakthroughs required to eradicate disease (e.g., cancer, cardiovascular disease, arthritis, etc.), to successfully program cells for therapeutic purposes (e.g., stem cells, cells involved in inflammation, carrier cells for nanoparticle delivery, etc.), to prevent organ transplant rejection (in the absence of immuno-suppressant drugs), to enhance biomaterial compatibility and function, and to eventually allow for the development of purely synthetic cells, organs, and potentially organisms.

It is well evident that limitations in our current understanding the cell surface prevent the prediction of how cells interact with man-made and biological interfaces. Therefore, in order to understand how cells interact with surfaces, measurements must be performed directly between living cells and the surfaces in question. Indeed, much of our current understanding of how cells interact with surfaces has evolved from cell adhesion measurements, which have a long history in biological sciences. However, the techniques most widely used to measure interactions between cells and surfaces suffer from significant limitations with respect to the dynamic force range over which they can measure cell-cell or cell-substratum interactions. Moreover, they lack the ability to adequately mimic the biophysical parameters of biologically relevant systems other than those experienced from fluid flow. By measuring interactions between living cells using atomic force microscopy (AFM) or other microcantilever based methods, many of the limitations current cell adhesion measurement technologies can be avoided. However, technical limitations in the fabrication of modified microcantilevers with attached living cells have restricted the development of this technology.

Simulating Cells in Suspension. Traditional protocols for confining suspension culture or detached cells from surfaces involve the use of antibodies for specific ligands (such as those of the CD family), the attachment to the gycocalyx using lectins or highly positively charged interfaces, or are bound to the cell surface by covalent bonds through reactive chemistry. All of these mechanisms of cell confinement are known to result in subsequent signal transduction and modified gene expression which may provide artifacts in the intended applications of the force sensors. Considerable disadvantages of these existing binding methods are their lack of universality (i.e., cells must express the necessary ligands or chemical functional groups in sufficient quantities for attachment), which constrains the applicability and the useful force range of the sensors.

Simulating Tissue Cultures or Colonies of Multiple Cells. The major barrier for the use of tissue culture cantilever probes for industrial and widespread application is difficulty in manufacture. For this reason only a limited number of publications appear in the literature, most notably that of Benoit in 2002 (Benoit, M., (2002) “Cell Adhesion Measured by Force Spectroscopy on Living Cells”, Methods in Cell Biology, 68:91-114). Benoit's describes the growth of cells onto colloidal probes by impinging cells through the culture liquid such that they bombard the surface of a particle attached to the free end of a cantilever. To increase the probability of attachment, the surface of the particles were modified or chosen to promote adhesion upon immediate cell contact. Considerable disadvantages of this approach is the low probability of cell attachment—on the order of one cell per twenty attempts for standard tissue culture materials (i.e., for standard polystyrene or glass surfaces using MET-5A mesothelial cells) and about one in every six attempts for fibronectin coated surfaces, both for an individual trained in the art.

It is now well understood that underlying surface or material modifications can largely influence the resulting cell surface expression and gene regulation. Therefore, it is desirable to develop efficient cell attachment methods for adherent cells that do not necessitate material modifications to enhance attachment probabilities. Considerable disadvantages of the existing fabrication methods that are independent of the underlying material are the tedium and the general inefficiency of the attachment protocols. Moreover, existing attachment methods are not easily automated.

Single Adherent Cells. In concurrence with the above discussion, facile and efficient methods that enable the attachment of single adherent cells to the end of a microfabricated cantilever, independent of the underlying material, have not been reported.

### SUMMARY

Disclosed herein are methods for the rapid and efficient attachment of living cells to microcantilevers. The methods developed have been designed to be facile and widely applicable to nearly all cell types. These developments are expected to bring widespread attention to the use of cantilever based detection systems for cell adhesion measurement and data mining applications, bringing forth new devices and biological insights.

Several methods are disclosed for depositing living cells at free end of microcantilevers for the simulation of distinct physiologically relevant states. An integrated device for fabricating living cell-terminated microcantilevers and measuring interaction forces between said cantilever and surfaces is presented. Also, disclosed are automated approaches for fabricating and applying such sensors for bioanalytical purposes.

In one embodiment for simulating cells in suspension, the free end of a microcantilever is functionalized with molecules containing a hydrophobic group and a hydrated spacer molecule. The cantilever is brought into contact with living suspension culture or detached adherent cell resulting in a self-assembled living cell force sensor. The resulting force sensor can be fabricated with any living cell containing an exterior lipid membrane. The strength of cell attachment to the cantilever is not dependent on the existence of specific receptors or chemically reactive groups on the cell surface. The strength of cell attachment and applicable dynamic range of the force sensor can be modified by controlling the number of functionalizing molecules, the length and composition of the spacer molecule, the hydrophobicity of the terminal hydrophobic group, and the bond strength between the cantilever and the spacer molecule. The strength of attachment can be modified to significantly exceed those obtained by using specific ligand-receptor bonds.

In another embodiment, the functionalized cantilever can be used to create force sensors terminated with other particles formed via hydrophobically driven self-assembly. Such particles could be emulsion droplets or liposomes. Said particles are preferably attached as whole particles and not spherical caps as reported by other methods. Attached particle and composite force sensors, therefore better represent the original particle system.

According to another embodiment for simulating tissue cultures or colonies of multiple cells, the free end of a cantilever is terminated by a large particle or microfabricated protrusion, preferably with an exposed convex surface. A hanging drop containing the living cells of interest is placed near the terminal feature of said cantilever in a gaseous environment. Cells of interest are transferred to the terminal feature utilizing capillarity. The cantilever(s) is then placed in suitable cell culture media to allow for adherent cells to spread and grow to the desired level of confluence. Such protocols result in cell attachment probabilities of greater than 80 to 90 percent success rates for individuals trained in the art.

According to another embodiment for simulating a single adherent cell attached to substrate, a cantilever is terminated by a large particle or microfabricated protrusion, preferably with an exposed convex surface. The surface of terminal feature is chemically modified with a highly hydrated surface molecular layer except at its apex. Cell attachment proceeds as discussed above. In another embodiment for simulating a single adherent cell attached to a substrate, a cantilever is selectively chemically modified with a hydrophobic agent such that the surface energy of the cantilever is reduced except at the working free end. The working free end of the cantilever is brought into contact with the hanging drop containing the cells of interest and subsequently removed.

In all embodiments described in this section, cantilever dimensions and spring constant can be manipulated to modify the sensitivity and applicable force range of the overall force sensor device.

### BRIEF DESCRIPTION OF THE DRAWINGS

FIG. 1. is a schematic, side cross-sectional view of a cantilever functionalized with the preferred embodiment of a molecule containing a hydrated and hydrophobic group.

FIG. 2. is a schematic, side cross-sectional view of a living cell force sensor of a preferred embodiment for simulating cells in suspension.

FIG. 3. Comparison of the proliferation of human peripheral monocytes (THP-1, American Type Culture Collection, Manassass, Va.) in RPMI 1640 media with 5% Fetal Bovine Serum under standard suspension culture conditions to those attached to a surface via a fatty acid terminated polyethylene glycol linkers as disclosed in FIGS. 1 and 2 above.

FIG. 4. Corresponding data (with respect to FIG. 3) comparing the viability of human peripheral monocytes (THP-1, American Type Culture Collection, Manassass, Va.) in RPMI 1640 media with 5% Fetal Bovine Serum under standard suspension culture conditions to those attached to a surface via a fatty acid terminated polyethylene glycol linkers as disclosed in FIGS. 1 and 2 above.

FIG. 5. is a schematic, side cross-sectional view self-assembled particle terminated force sensors containing a single particle.

FIG. 6. is a schematic, side cross-sectional view of a living cell force sensor of an embodiment for simulating multiple suspension culture cells. Alternatively, such a sensor may be fabricated to contain multiple particles (e.g., emulsion droplets or drug delivery liposomes).

FIG. 7. is a schematic, side cross-sectional view of a living cell force sensor of an embodiment for simulating tissue cultures or surface colonies of multiple cells. In this version of living cell force sensors, the cells are allowed to grow to enable the presentation of phenotypic expression resulting from cell-surface interactions. Note, in the embodiment schematically depicted in FIG. 4, the hydrated spacer molecule, inhibits cell surface interactions, such a coating is not used in the present case.

FIG. 8. is a schematic, side cross-sectional view of an embodiment for cell seeding based on capillary wetting induced by drop advancement and retraction.

FIG. 9. is a schematic, side cross-sectional view of preferred embodiment for cell seeding based on capillary wetting induced by normal translation.

FIG. 10. is a sequence of images (left to right) exemplifying the process in the schematic given as FIG. 9. In this example the drop reservoir is translated to contact and disengage with the colloidal probe.

FIG. 11. is a schematic, side cross-sectional view of another embodiment for cell seeding based on capillary wetting induced by lateral translation.

FIG. 12. is a sequence of images, illustrating the embodiment described in schematic given in FIG. 11.

FIG. 13. is a schematic, side cross-sectional view of a preferred embodiment for cell seeding based on capillary wetting induced by an electric potential.

FIG. 14. is a sequence of images (a-f) illustrating capillary cell transfer induced by applied electric potential, schematically illustrated in FIG. 13, the process is shown under lateral translation to demonstrate the long range attraction induced by the electric potential.

FIG. 15. presents optical micrographs of (a) a colloidal probe prior to cell seeding, (b) the same colloidal probe illustrated in (a) after lateral translation induced cell seeding (shown in FIG. 10.) using a 1000 MET-5A human mesothelial cells in growth media, (c) a similar colloidal probe as given in (a) and (b) seeded by electric potential induced capillary transfer under identical cell loading conditions (shown in FIG. 14). All images are of the same scale.

FIG. 16. is a chart illustrating the differential success rate between the standard and the disclosed cell attachment method for MET-5A human mesothelial cells on polystyrene microsphere-terminated cantilevers. For each cell concentration and method, twenty attempts were made. The results indicate the percentage of cantilevers with at least one attached cell out of twenty trials for each seeding technique and total cell concentration. Note that the standard impingement method refers to the method where cells are injected towards a microparticle immersed in growth media.

FIG. 17. is a schematic of an automated device for cell seeding based on capillary wetting. A similar manual device was used in FIGS. 10, 12, and 14.

FIG. 18. Schematic of an integrated device for fabricating living cell-terminated microcantilevers and measuring interaction forces between said cantilever and test surfaces is presented. In this view, a mode suitable for capillary transfer of living cells onto cantilevers is presented.

FIG. 19. Schematic of an integrated device for fabricating living cell-terminated microcantilevers and measuring interaction forces between said cantilever and test surfaces is presented. In this view, a mode suitable for measuring interaction forces between cell probes and test substrates is presented.

FIG. 20. Schematic of an integrated device for fabricating living cell-terminated microcantilevers and measuring interaction forces between said cantilever and test surfaces is presented. In this view, an alternative mode suitable for measuring interaction forces between cell probes and test substrates is presented.

FIG. 21. Schematic side cross-sectional view of one embodiment of a simple diagnostic device utilizing living cell force sensors.

FIG. 22. Schematic of an automated device for using said living cell terminated cantilevers for high throughput screening for cell-surface ligands or other potentially biologically active compounds associated with the microarray. Alternatively, the plate can be automated.

FIG. 23. Schematic of Living Cell Force Sensor, a selection of available motifs and general scheme of implementation in AFM/SPM.

FIG. 24. Illustration of an example of the universal single-cell probe (not drawn to scale). Note: This method allows for the passive constraint of cells to simulate their behavior as if they were suspended in biological media and not attached to a surface. The noted moieties may be substituted for other similar groups.

FIG. 25. contains a schematic of the standard impingement seeding process and a chart indicating the relative probability of attaching MET-5A human mesothelial cells to microparticle terminated cantilevers, with and without the use of fibronectin as an adhesion modifier.

### DETAILED DESCRIPTION

It is well evident that limitations in our current understanding the cell surface prevent the prediction of how cells interact with man-made and biological interfaces. Therefore, in order to understand how cells interact with surfaces, measurements must be performed directly between living cells and the surfaces in question. Indeed, much of our current understanding of how cells interact with surfaces has evolved from cell adhesion measurements, which have a long history in biological sciences. The measurement of cell adhesion, or cell interaction forces, can be critical for the early diagnosis of disease, the design of targeted drug and gene delivery vehicles, the development of next-generation implant materials, and much more. However, the technologies and devices that are currently on the market are highly limited with respect to the dynamic force range over which they can measure cell-cell or cell-substratum interactions, and with their ability to adequately mimic biologically relevant interactions (Table 1). Consequently, research that involves cell adhesion has been technologically limited.

TABLE 1
Cell adhesion measurement techniques and applicable force ranges

| Technique | Force Range | Comment |
|---|---|---|
| Aggregation Assays | Yes/No | No force information |
| Plate & Wash | Yes/No | No force information |
| Centrifugation | 15-150 pN/Cell | Inability to determine origin of |
| | | force, prone to nonspecific |
| | | artifacts |
| Hydrodynamic | 500-1000 pN/Cell | Laminar flow required |
| Techniques | | |
| TIRM, | 10 fN-1000 pN/Cell | Limited to single suspension |
| EWLS-3DOT | | culture cells, Rotational |
| | | freedom |
| Microfabricated | 1 pN->1000 mN/Cell | Identification of origin of force |
| Cell-based Sensors | | possible through fingerprinting |
| with a conventional | | Mechanical simulation of |
| AFM | | physiological environment |
| | | possibleAlso allows for nanoscale |
| | | imaging, force mapping, etc. |

By measuring interactions between living cells using atomic force microscopy (AFM) or other microcantilever based methods, many of the limitations current cell adhesion measurement technologies can be avoided. However to date, technical limitations in the fabrication of modified microcantilevers with attached living cells have restricted the development of this technology.

To meet the current scientific needs, the inventors have utilized our background in nanoscience to develop improved protocols and devices for the rapid fabrication of living cell force sensors technologies (FIG. 23). These sensors allow for the highly sensitive measurement of cell-mediated interactions over the entire range of forces expected in biotechnology (and nano-biotechnology) research (from a single to millions of receptor-ligand bonds). In tandem, with cell seeding method embodiments, several force sensor motifs have been developed that can be used to measure interactions using single adherent cells, single suspension culture cell, and cell monolayers (tissues) over a wide range of interaction conditions (e.g., approach velocity, shear rate, contact time, etc.). Hence, the inventors have created a unique system to provide tools for studying changes in cell adhesion behavior as a function of confluency, differentiation, and other highly important environmental and physiological factors that until now, were not easily achieved.

The fabricated cell force sensors are consumables that essentially convert conventional atomic force microscopes (AFMs), or scanning probe microscopes (SPMs), into highly sensitive, robust, and unique cell adhesion/interaction force measurement device. By streamlining methods for creating living cell probes for use in AFMs/SPMs, the widespread use of Cellular Probe Force Microscopy, a new analytical tool with unprecedented flexibility, sensitivity and multiple advantages over the state-of-the-art technologies on the market, is enabled. Recognizing that these probes could also be a valuable resource for the study of cell adhesion without an AFM or SPM tool, simpler devices that are designed to facilitate both the fabrication of cellular probes and their application for sample measurement have also been developed.

Method embodiments for production of these probes are non-intuitive and rely on a strong background in surface science for conceptualization. The inventors have discovered and developed capillary transfer techniques for the attachment of cells to cantilevers, which dramatically enhances their attachment efficacy and, is suitable for the large scale manufacturing of these probes. This protocol can be applied for both single cell and tissue style probes. Essentially the cells are suspended in a drop of media and the surface tension of that drop, in addition to its ability to wet the cantilever or particle surface, is used to confine the cells in close proximity to the intended surface for attachment.

In order to use microcantilevers to measure the interactions forces between cells that typically suspended in media, e.g., suspension culture cells or simulated detached adherent cells, in one embodiment, a unique method has been developed that can be universally applied to strongly and passively adhere them to microcantilever surfaces. By using a hydrophobic molecule of similar characteristics to the cell membrane, attached to a bio-inert spacer molecule (e.g., polyethylene glycol or other suitable spacer as will realized by the teachings herein), the inventors have been able to constrain cells to cantilevers with very little impact on their function. Conventional techniques target either sugar molecules on the cell surface or other specific receptors and therefore are subject to artifacts from subsequent signal transductions and changes in gene regulation.

According to one embodiment for simulating cells in suspension, the free end of a microcantilever is functionalized with molecules containing a hydrophobic group and a hydrated spacer molecule. The length of the spacer molecule is at least 10 nm and preferably 50 to 500 nm. The spacer molecule may be composed of polyethylene glycol, carbohydrates, or other highly hydrated hydrogen bonding materials. The hydrophobic group is attached to the free end of the spacer molecule. Preferably, the hydrophobic group consists of a fatty acid, phospholipid or cholesterol. Alternatively, the hydrophobic group consists of a synthetic surfactant with a critical micelle concentration between 10⁻² and 10⁻⁹M and is preferably unsaturated. Said cantilever is brought into contact with living suspension culture cell or detached adherent cell resulting in a self-assembled living cell force sensor. The proliferation and viability of cells on said force sensor is comparable to that of the free cells in suspended in culture media.

The resulting force sensor can be fabricated with any living cell containing an exterior lipid membrane. The strength of cell attachment to the cantilever is not dependent on the existence of specific receptors or chemically reactive groups on the cell surface. The strength of cell attachment and applicable dynamic range of the force sensor can be modified by controlling the number of functionalizing molecules, the length and composition of the spacer molecule, the hydrophobicity of the terminal hydrophobic group, and the bond strength between the cantilever and the spacer molecule. The strength of attachment can be modified to significantly exceed those obtained by using specific ligand-receptor bonds.

In another embodiment, the functionalized cantilever can be used to create force sensors terminated with other particles formed via hydrophobically driven self-assembly through an identical micromanipulation driven, self-assembling attachment process. Such particles could be emulsion droplets or liposomes. Said particles will be attached as whole particles and not spherical caps as reported by other methods. Attached particle and composite force sensors, therefore better represent the original particle system.

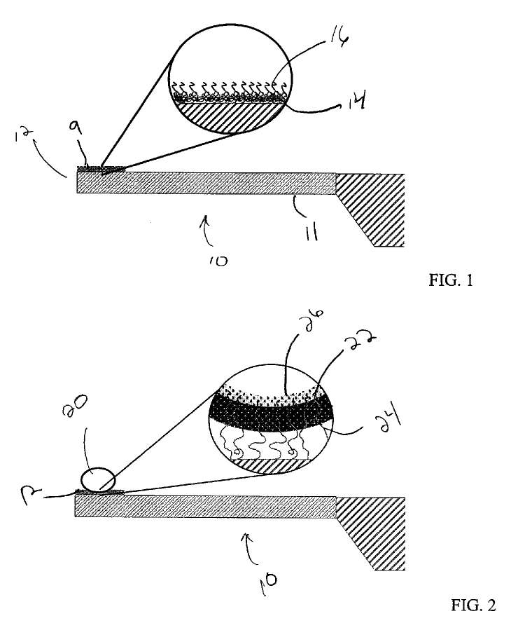

FIG. 1 shows a cantilever 10 with an arm 11 (or lever portion) with a probe portion 9 provided at the free end 12 that has been functionalized to include a hydrophobe layer 16 wherein the hydrophobe is attached to a spacer molecule layer 14. FIG. 2 shows a cell 20 attached to the functionalized free end 12 of the cantilever 10. The close up shows the cell membrane 26 with hydrophobe molecules 22 interacting therewith and spacer molecules 24 attached to the hydrophobes molecules 22.

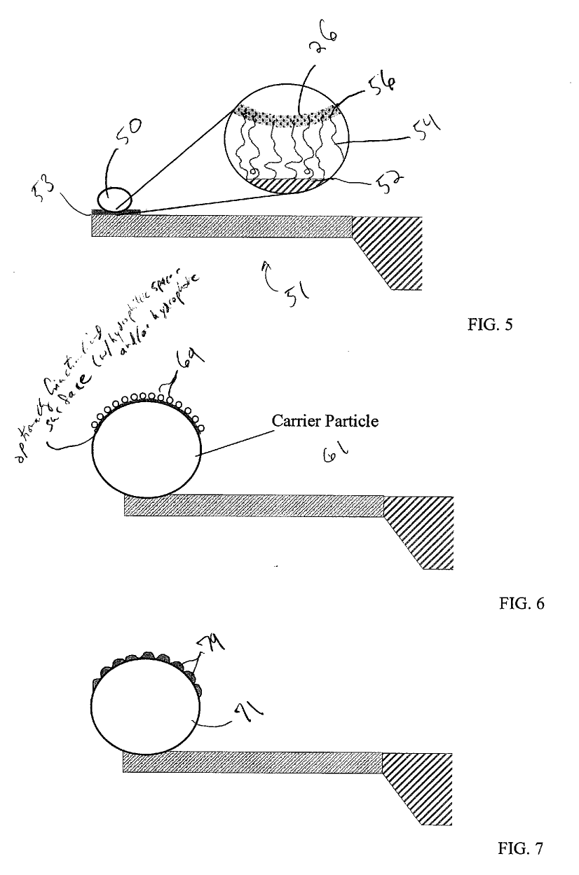

FIG. 5 shows a cantilever 51 with a self-assembling particle 50 associated with the free end 53 of the cantilever 51. The particle 50 has a hydrophobic layer with which hydrophobe molecules 56 are associated. The hydrophobe molecules 56 are conjugated to spacer molecules 54, which in turn are associated with the surface of the free end 53.

Conventional techniques are limited to cells that have the appropriate receptors for the ligands used, and an adequate number of ligands present to impose enough of an attachment force. (note: the forces measured by the cantilevers are limited by the force attaching the cell to the cantilever) By using a hydrophobic lipid or lipid-like anchor attached to a spacer molecule (that allows penetration into the cell coat) the inventors have developed a unique, universal and perhaps the strongest means by which one can passively attach a living cell to a cantilever. The characterization of ‘strongest’ is used because ultimately all receptors and molecules on the cell surface are associated with the lipid bilayer. Therefore, the force holding them to the bilayer is effectively the limiting force that can be used to attach anything to the cell. By directly integrating into the bilayer (cell membrane), embodiments of the invention are capable of directly tapping into this very strong binding mechanism. Moreover, because the ligand goes directly to the bilayer using solely hydrophobic interactions, it is believed that attachment mechanism embodiments do not lead to any adverse signal transduction.

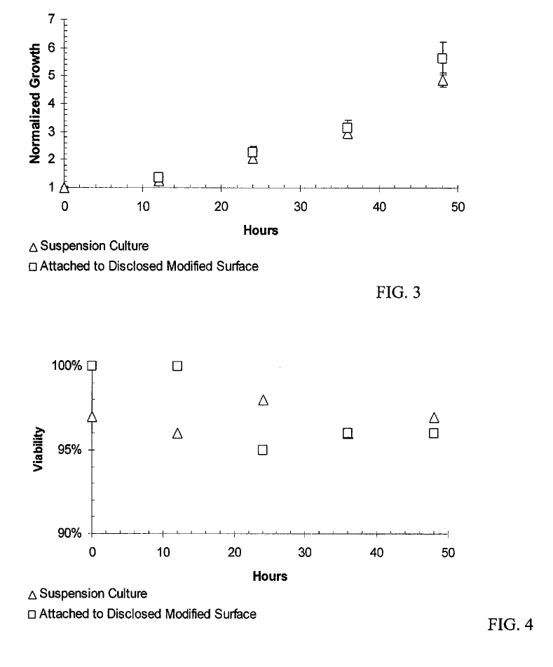

FIG. 3 shows a comparison of the proliferation of human peripheral monocytes (THP-1, American Type Culture Collection, Manassass, Va.) in RPMI 1640 media with 5% Fetal Bovine Serum under standard suspension culture conditions to those attached to a surface via hydrophobe (e.g. fatty acide) terminated spacer molecules (e.g. PEG or other suitable spacer) as disclosed in FIGS. 1 and 2 above. FIG. 4 shows corresponding data (with respect to FIG. 3) comparing the viability of human peripheral monocytes (THP-1, American Type Culture Collection, Manassass, Va.) RPMI 1640 media with 5% Fetal Bovine Serum under standard suspension culture conditions to those attached to a surface via a hydrophobe terminated spacer molecules as disclosed in FIGS. 1 and 2 above.

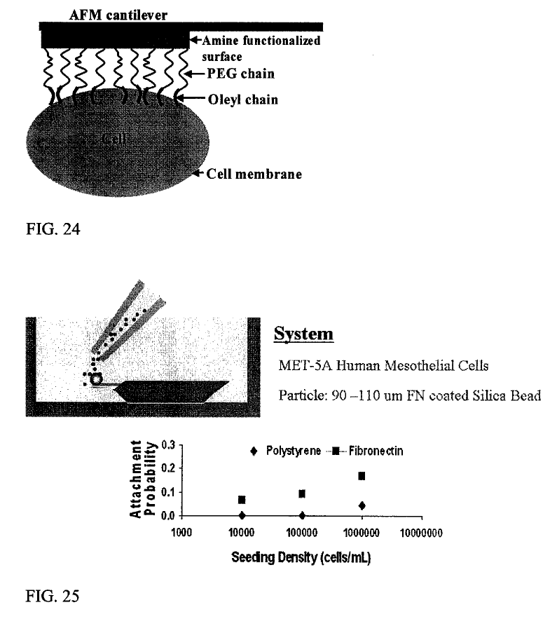

In a specific embodiment, cantilevers have been surface-functionalized with amine groups and subsequently reacted with an oleylo-o-poly(ethylene glycol)-succinyl-N-hydroxy-succinimidyl (NHS) ester. The NHS group of this ligand is used to covalently bind to the amine groups on the probe surface whereas the free oleyl group is used to passively bind to the cell membrane (see FIG. 24). Polyethylene glycol (PEG) is used as a spacer molecule to prevent ‘extra’ surface interaction between the attached cell and probe and to penetrate the cell coat. Note: without the PEG spacer attachment does not occur. This oleyl group based cell immobilization method has been used to immobilize nonadherent cell lines onto planar substrates with no noticeable changes to modifications to cell viability or proliferation rate. With respect to the attachment protocol, a single cell is attached to the end of the cantilever via micromanipulation prior to experimentation or by capillary transfer. Those skilled in the art in view of the teachings herein will appreciate that other spacer molecules may be used including, but not limited to, polyoxyethylene, polymethylene glycol, polytrimethylene glycols, polyvinyl-pyrrolidones, polyvinyl alcohol, polyvinyl pyrrolidone, polyethylene oxide, and derivatives thereof. The polymers can be linear or multiply branched.

The innovative embodiment shown in FIG. 24 provides the skilled artisan with a large degree of freedom and opportunities not enabled by conventional in vitro SPM. By placing the cells of interest on the force sensor and not on the planar substratum, one is afforded the ability to scan cellular interactions with spatially resolved multiple domains of varying architecture (chemical and/or structural properties), thereby allowing assessment of multiple well-defined regions in a single experiment.

By using these probes, one can now datamine surfaces containing arrays of proteins and potential drug targeting molecules (or molecules of other intended uses). The importance of this technique for drug discovery may be immense. Currently cell membrane proteins account for 70% of all known pharmaceutical drug targets, and 25% of these are class 1 and class 2 GPRCs. Pharmaceutical cell surface targets have been limited since many cell surface proteins and their functions are unknown. Here the inventors provide a technique where you don't need to know the proteins on the surface, per se, but will be able to detect if a unique binding event occurs. Hence it provides a way to rapidly screen for unknown molecular scale binding between cells and a variety of molecules (i.e., target identification) thereby allowing for the cell surface to be datamined for new pharmaceutically or bioanalytically relevant binding pairs.

In other embodiments of the invention, methods are provided by which cells can be easily seeded onto microcantilevers to create living cell force sensors that may or may not be utilized in an AFM. The methods disclosed are more efficient that those previously disclosed and can be applied effectively for very low cell concentrations. By making probe creation simple and possible when only a small population of cells are available, living cell force sensors become a viable option for bed side diagnostics especially in the many cases where cell surface interactions are important. Such cantilever systems may be integrated into devices that can be used to replace current calorimetric and fluorescence based kits which require costly consumable reagents. By using a cantilever system for reading the presence of a particular antibody or other cell surface molecule, one can take advantage of the reversibility of the specific binding interactions found in biology to fabricate a reusable device. For instance, antibodies, aptamers, or other ligands may be constrained to a surface that preserves their shelf life and also allows for them to be brought into contact and detached from said cell sensors resulting in an obvious change in cantilever bending. Alternatively, these sensors can also be used in proteomics and other data mining applications where molecular units on the cell surface may be of interest. Such an application could be, for example, the search of a new ligand for targeting a particular type of cancer cell. Because a very limited number of molecules on the cell surface are known, and less are known under any given environmental condition, through the use of living cell microcantilever systems one has the unique opportunity to begin to identify important ligands prior to understanding the nature of cell surface receptors involved. In other words, one can potentially used the disclosed microcantilever systems to identify and procure targeting ligand for cells under different environmental states, thereby identifying new routes for therapeutics as well as understanding the process that undergo at the cell surface. Examples of devices that can be used for the application of said force sensors, other than typical AFMs, are also disclosed herein.

In a specific embodiment for simulating tissue cultures or colonies of multiple cells, the free end of a cantilever is terminated by a large particle or microfabricated protrusion, preferably with an exposed convex surface. The diameter or effective width of the terminal feature is at least 20 microns, preferably 100-500 microns. A hanging drop containing the living cells of interest is placed near the terminal feature of said cantilever in a gaseous environment. In an alternative embodiment, an electrical charge is applied to cause the cantilever to bend into said hanging drop, causing the formation of a capillary bridge with the terminal feature. Subsequently, charge dissipation causes the cantilever to detach from the surface resulting in capillary transfer of cells of interest to the apex of the terminal feature. In another embodiment, the drop and terminal feature are brought into contact by micromanipulation then disengaged to invoke capillary transfer. In still another embodiment the hanging drop is placed adjacent to the terminal feature and lateral translation is used to bring one or more terminal features attached to separate cantilevers into capillarity with the hanging droplet. Lateral translation also results in capillary transfer of the cells of interest. Subsequent to transfer for the above method embodiments, the cantilever(s), optionally, may then be placed in suitable cell culture media to allow for adherent cells to further spread and grow to the desired level of confluence. The probability of attachment using said methods is better than eight in every ten trials for a person trained in the art.

FIG. 6 shows a cantilever with a carrier particle 61 onto which cells 69 have been disposed. Alternatively, all or a portion of the surface of the particle 61 may be functionalized as described above. FIG. 7 is a schematic, side cross-sectional view of a living cell force sensor of an embodiment for simulating tissue cultures or surface colonies of multiple cells. In this version of living cell force sensors, the cells 79 are allowed to grow on the particle 71 to enable the presentation of phenotypic expression resulting from cell-surface interactions. Note, in the embodiment schematically depicted in FIG. 24, the hydrated spacer molecule, inhibits cell surface interactions, such a coating is not used in the present case.

In another embodiment for simulating tissue cultures or colonies of multiple cells, the terminal feature may be left immersed in the hanging drop containing the cells of interest and incubated under suitable cell culture environment to allow for enhanced attachment. Such protocols can result in attachment probabilities better than nine in every ten trials for a person trained in the art.

In a specific embodiment for simulating a single adherent cell attached to substrate, a cantilever terminated by a large particle or microfabricated protrusion, preferably with an exposed convex surface. The diameter or effective width of the terminal feature is at least 10 microns, preferably 75-200 microns. The surface of terminal feature is chemically modified with a highly hydrated surface molecular layer except at its apex. The surface molecular layer may be composed of polyethylene glycol, carbohydrates, or other highly hydrated hydrogen bonding materials. Cell attachment proceeds as discussed in the previous two sections.

In another embodiment for simulating a single adherent cell attached to a substrate, a cantilever is selectively chemically modified with a hydrophobic agent such that the surface energy of the cantilever is reduced except at the working free end. The surface energy differential between the hydrophobically modified portion and the remainder of the cantilever is sufficient enough to invoke selective wetting of the working free end. The working free end of the cantilever is brought into contact with the hanging drop containing the cells of interest and subsequently removed.

In all embodiments described in this section, cantilever dimensions and spring constant can be manipulated to modify the sensitivity and applicable force range of the overall force sensor device.

### Suspension Culture or Simulated Detached Cells

Traditional protocols for confining suspension culture or detached cells from surfaces involve the use of antibodies for specific ligands (such as those of the CD family), the attachment to the gycocalyx using lectins, or are bound by covalent bonds through reactive chemistry. All of these mechanisms of cell confinement are known to result in subsequent signal transduction which may provide artifacts in experiments. Because so little is known with respect how signal transduction from these binding processes can alter the cell adhesion processes that are to be measured, methods that are less susceptible to these artifacts are desired. In order to fulfill this need the inventors have developed a unique method that mimics natural cellular processes to provide a passive yet strong attachment of a wide range of cells to interfaces.

Recognizing that lipids from the bulk fluid phase are constantly exchanged with the outer cell membrane in biological fluids, the inventors explored the possibility of using similar lipid-lipid interactions to attach cells to surfaces. Such interactions would mimic natural exchange processes that occur at the cell surface, thereby being more passive than traditional protocols, and also could be widely applied and tuned to nearly every cell type.

In biological systems, individuals trained in the art often regard covalent bonding and subsequently receptor-ligand interactions as the strongest forms of binding found in biological systems. However, these assumptions are based on interferences from classical texts, which often underestimate the interactions between hydrophobic chains as being purely based on van der Waals attraction between the hydrocarbon chains and tend to neglect the complex response of water to hydrophobic surfaces. Indeed, in even the most popular of texts including the fifth edition of Molecular Biology of the Cell, hydrophobic forces are mentioned but not indexed as one of the strongest binding forces in biological systems. The obscurity of this information, even to those trained in the art, results from a general, relatively poor understanding of the complex phenomena that invoke hydrophobic bonding interactions.

Recognizing that most cell surface receptors are primarily tethered to the cell via hydrophobic interactions with the phospholipid bilayer, a simple engineering analysis suggests that the ultimate binding strength of any receptor-ligand bond is a function of the weakest link. Hence, upon application of a pulling force, either the receptor-ligand bond will break or the receptor will be pulled out of the phospholipid membrane. As mentioned previously, hydrophobic interactions between molecules are difficult to calculate due to the contribution of solvent interactions that are not well understood. Hence, experimental measurements provide the most representative data. Single molecule hydrophobe interactions have been reported in the literature for 18-carbon saturated alkyl chains interacting with an opposing monolayer contain the same hydrophobe. The measured pull-off forces were in the range of 600-700 pN across water. Recalling that most single receptor-ligand bonds undergoing similar unbinding kinetics are of the order of 50-200 pN per bond, it is likely that hydrophobic interactions could dominate in many scenarios. Another consideration to keep in mind is that the total attachment force for cells to surfaces is related to both the number and strength of the respective binding interactions. If one were to attach cells to cantilevers using receptor-ligand bonds, the maximum attachment force would ultimately be limited by the number of attachment sites on the cell. By using hydrophobic binding interactions rather than receptor-ligand bonds, this limitation is avoided.

From the above discussion, it is evident that hydrophobic binding could provide a robust means for attaching single suspension culture cells to cantilever surfaces. However, the question remains on how to design an effective hydrophobe anchor. There are at least two considerations to take into account when attempting to integrate hydrophobes into living cell membranes. The first is the normal thermal residence time of hydrophobe in the phospholipid bilayer and the second is the tendency for the hydrophobe to associate with phase separated domains in the bilayer, which could also lead to signal transduction. The former will limit the minimum rate of force measurement, whereas the later will define both the upper limiting magnitude of force measurement per molecule as well as the structure of the hydrophobe. Hence insights into the design of the hydrophobic portion of lipid anchors can be taken from their estimated residence time in self-assembled structures in addition to their chain melting temperature.

In essence, the exchange of monomer to the bulk solution is an activation process in which activation energy (ΔE) must be surpassed for before a molecule can escape from the bilayer to the bulk solution. The probability of a molecule leaving the bilayer each time it moves towards the interface is effectively given by e^{-ΔE/kT}, where k is the Boltzman constant and T the temperature of the system. Considering that there must be a characteristic time, τₒ, at which the phospholipids collide towards the interface, then the residence time of a lipid in a bilayer can be represented as Eq. 1-1.

$${τ}_{R} = \frac{{τ}_{0}}{{e}^{- Δ E / kT}} \qquad ( 1 \text{-} 1 )$$

Theoretically, the activation energy should be similar to the difference in the standard chemical potential (the mean interaction energy per molecule) between molecules in the monomer state, μo₁, to that of those in the equilibrium bilayer structure, μo_{N}, as given by Eq. 1-2.

ΔE=(μ₁⁰−μ_{N}⁰) (1-2)

From the fundamental thermodynamic equations of self assembly (Nagarajan and Ruckenstein, 1977; Nagarajan and Ruckenstein, 1979; Nagarajan and Ruckenstein, 1991), the critical micelle concentration can be approximated as given by Eq. 1-3.

$$C M C \approx exp [ \frac{- ( {μ}_{1}^{0} - {μ}_{N}^{0} )}{kT} ] \qquad ( 1 \text{-} 3 )$$

Therefore, τ_{R} can further be estimated as Eq. 4-4.

$${τ}_{R} \approx \frac{55 {τ}_{0}}{C M C} \qquad ( 1 \text{-} 4 )$$

Given the typical motional correlation times for amphiphiles in micelles and bilayers (τ₀) is found to be the range of 10⁻⁹-10⁻⁷ for surfactants in bilayers (Israelachvili, 1991) then the residence time, τ_{R}, for a typical hydrophobes can be estimated based on their pure system CMC. It should be noted that the rate of exchange of a single molecule is not significantly modified by its surrounding surfactants.

From the above discussion it becomes apparent that the best suited hydrophobe would have both a low CMC and low chain-melting temperature. The introduction of a double bond, or unsaturation in the hydrophobic chain can allow for both low CMCs and low chain melting temperatures. Moreover, the anchoring strength of the hydrophobe can be further increased by using a double chain. If one looks towards the composition of the lipid bilayer, it is well evident that nature uses both of these design criteria for the bulk of the lipid bilayer structure. Most phospholipids are double-chained with one unsaturated to give both fluidity and high bilayer residence times. Considering that the CMC of phospholipids range between 10⁻⁸-10⁻¹⁰ M their estimated bilayer residence time is in the range of 10¹ to 10⁴s, which is several orders longer than most single chained surfactants.

However, in order to prepare effective hydrophobic anchors, more considerations need to be taken. Experimental force curves using an alkylsilane modified AFM tips showed no adhesion to the surface of human mesothelial cells. Most cells are coated by sugar residues, collectively known as the glycocalyx. Because the length scales of these molecules are of the order of tens to several hundred nanometers thick, they act as a steric repulsive barrier and inhibit the interaction of hydrophobic moieties with the cell surface. At most, the alkane silanes used in the experiments were of approximately 3 nm in length, hence it became clear that longer molecules needed to be employed to reach the plasma membrane.

Selecting longer hydrophobic chains would typically not be desired, simply because they would lack the fluidity necessary for passive integration into the cell membrane. Instead, the inventors opted to use a highly hydrophilic spacer molecule which mimics the properties of the sugar residues that natively reside at the cell surface. Polyethylene glycol, sugar residues and other highly hydrated molecules are believed to provide the necessary properties for transcending the glycocalyx. These molecules would also allow for near normal transport of ions and other water soluble entities towards the constrained cell surface. By using a hydrophilic spacer molecule attached to the surface of the free end of a cantilever and terminated with a fatty acid hydrophobe, the inventors were able to successfully attach multiple human cell types to AFM cantilevers by simply positioning the cells under the tip and engaging the surface with the cantilever.

The force required to remove the cell from the cantilever was found to be in the vicinity of several hundred mN/m, which is much stronger than the current attachment methods used in the literature (generally in the 1-10 mN/m range), and therefore allows for these types of cantilevers to be used for the study of a wider range of bonding interactions.

To ensure that the cantilevers do not significantly impact the viability of suspension culture cells the inventors compared the proliferation of human peripheral monocytes (THP-1, American Type Culture Collection, Manassass, Va.) on PEG-fatty acid terminated surfaces to those cultured in bulk suspension. No apparent differences in growth rate or viability were found. When detached adherent cells were grown on the surfaces, the inventors notice that their viability decreased dramatically within 24 hours. The death pathway is believed to be anoikis since the cells were unable to attach to the surface using native adhesion molecules as apparent by their inability to obtain a non-spherical morphology. By using highly hydrophilic spacer molecules attached to a membrane inserting hydrophobe, it appears that the inventors can tether cells to surfaces in a manner which mimics their behavior in the bulk. In essences the hydrophilic spacer molecules not only allow insertion of the hydrophobe into the lipid membrane but also provide a cushion that inhibits significant intermolecular artifacts from being proximal to a surface.

### Tissue Culture or Adherent Cells

The major barrier for the use of tissue culture cantilever probes for industrial and widespread application is difficulty in manufacture. For this reason only a limited number of publications appear in the literature, most notably that of Benoit in 2002 (Benoit, M., (2002) “Cell Adhesion Measured by Force Spectroscopy on Living Cells”, Methods in Cell Biology, 68:91-114). In Benoit's article he describes the growth of cells onto colloidal probes. Recognizing the tedium involved it is explicitly mentioned that multiple trials are necessary to facilitate the attachment of enough cells which could then be grown into a monolayer on a single cantilever probe. In the published approach the cells were impinged through liquid onto the surface of a particle attached to the free end of a cantilever. To increase the probability of attachment, the surface of the particle was modified or chosen to promote adhesion upon immediate cell contact. Because this method relies on surface modification or the selection of alternative materials to attach adherent cell lines its applicability is limited. It is now well recognized that subtle changes in the surface properties of scaffolds or cell culture materials can have a dramatic impact on cell growth and gene expression.

Previously the inventors have independently attempted similar methods related to that as described by Benoit, and found very low attachment probabilities on the order of one success in every twenty trials. By the addition of an adhesion modifier (fibronectin in this case) the probability for the attachment of enough cells to allow monolayer growth only increased to about 1 success in about every six trials for an individual trained in the art.

Considering the forces involved in the standard seeding process, the inventors realized that hydrodynamic effects could deform the cell surface upon sedimentation of the particles to the surface. Because the duration of the time of approach for cells seeded via pipetting is rather small (i.e., less than a second—reflecting the time of initial close approach together with the time required for the cell to slide away from surface) deformation of the cell surface through the conventional impingement approach could mitigate cell surface contact, hence significantly lowering contact probability. Simply, as cells are forced towards a surface, their surface deforms upon close approach due to their low surface tension which—because of the magnitude of forces involved—is more likely to maintain a separation distance rather than the squeezing out the stagnant fluid layer that resides close to the surface to enable cell-surface contact.

Considering this phenomenon, the inventors hypothesized that improving the residence time of the cells to the surface could substantially improve the seeding probability of colloidal probes for tissue simulating cantilever production. Ultimately, it is desirable to create a method that is simple, relatively quick, and robust enough to not be adherent cell line or material dependant. Moreover, developing a method that is easily adaptable to automation would be critical if these sensors are to be used in high throughput applications.

Realizing that the curvature of a microparticle, could be used to prevent liquid wicking onto the supporting cantilever, the inventors attempted to seed cells onto microparticle terminated cantilevers by simply partially wetting a large microparticle attached to a cantilever with a hanging drop containing the cells of interest. By doing this, it was found that the cells could be easily confined to the surface of microparticle within a few seconds to minutes depending on the seeding parameters. In addition, it was found that by applying a bias between the droplet containing the cells of interest and the cantilever that the inventors could simply move the cantilever under the drop and the cantilever would bend upwards, automatically dipping into the cell laden drop. Both approaches had success rates greater than nine out of every ten trials. Moreover the latter two approaches are amicable to automation. In addition to ease of attachment, the direct seeding of cells at the apex of a microparticle attached to the end of the cantilever, also mitigates the probability for cells to attach to the cantilever beam surface-potentially interfering with optical cantilever deflection detection systems. Hence, by using this method one also avoids the need for chemical modification steps to prevent cell attachment to the cantilever beam (e.g., by applying a layer of PEG).

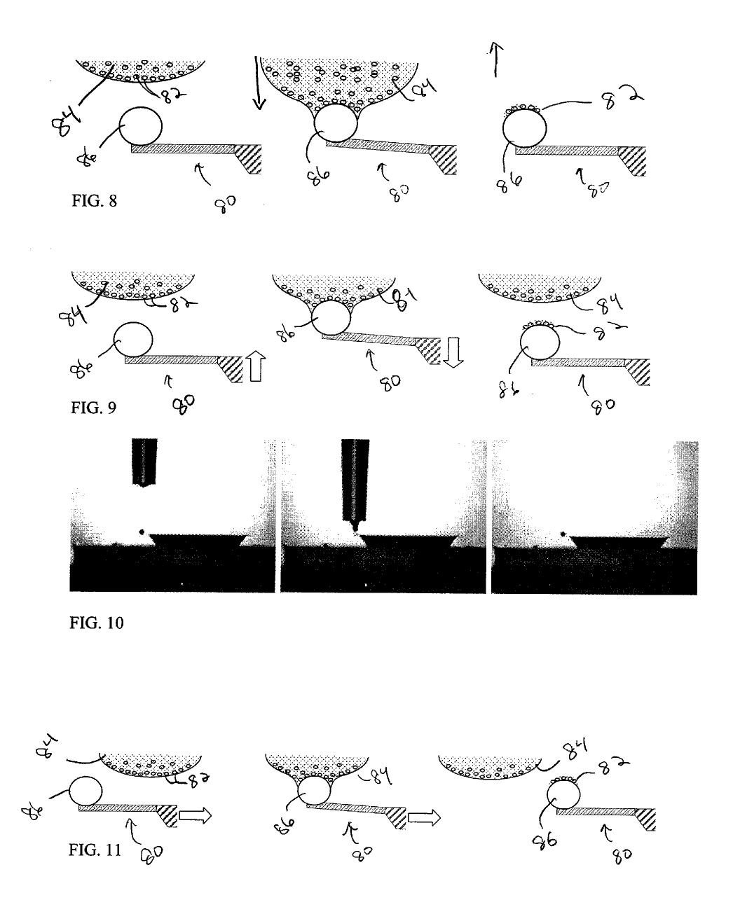

FIG. 8. shows a schematic, side cross-sectional view of an embodiment for cell seeding based on capillary wetting induced by drop advancement and retraction. A droplet of media 84 containing cells 82 is lowered onto a particle 86 associated with a cantilever 80. The droplet 84 is then raised off of particle 86 thereby leaving cells 82 associated with the particle 86.

FIG. 9 shows a schematic, side cross-sectional view of an embodiment for cell seeding based on capillary wetting induced by normal translation. A cantilever 80 having a particle 86 associated therewith is raised to come into contact with a droplet of media 84 containing cells 82. The cantilever 80 is lowered from the droplet 84 and cells 82 are left disposed onto particle 86. FIG. 10 shows s a sequence of images (left to right) exemplifying the process in the schematic given as FIG. 9. In this example the drop reservoir is translated to contact and disengage with the colloidal probe.

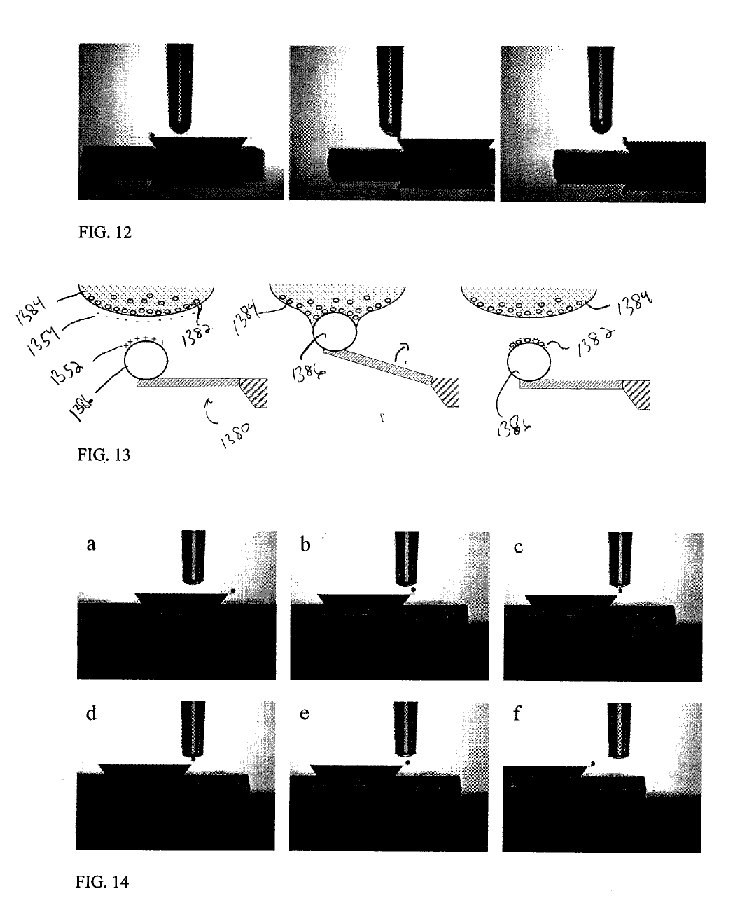

FIG. 11 shows a schematic, side cross-sectional view of another embodiment for cell seeding based on capillary wetting induced by lateral translation. In this embodiment, a cantilever 80 having a particle 86 associated therewith is laterally moved to bring the particle 85 against and into contact with a droplet 84 containing cells 82. The cantilever 80 is moved passed the droplet 84 thereby leaving cells 82 disposed on said particle 86. FIG. 12 is a sequence of images, illustrating the embodiment described in schematic given in FIG. 11.

FIG. 13 shows a schematic, side cross-sectional view of an embodiment for cell seeding based on capillary wetting induced by an electric potential. According to this embodiment, a cantilever 1380 having a particle 1386 with a positively charged surface 1352 is brought into proximity with a droplet of media 1384 containing cells 1382 and which is negatively charged. Due to attractive forces the cantilever arm flexes up to bring the particle 1386 into contact with the droplet 1384. Following this, the cantilever arm returns to its unflexed position whereby cells 1382 are disposed onto the particle 1386. FIG. 14 shows a sequence of images (a-f) illustrating capillary cell transfer induced by applied electric potential, schematically illustrated in FIG. 13, the process is shown under lateral translation to demonstrate the long range attraction induced by the electric potential.

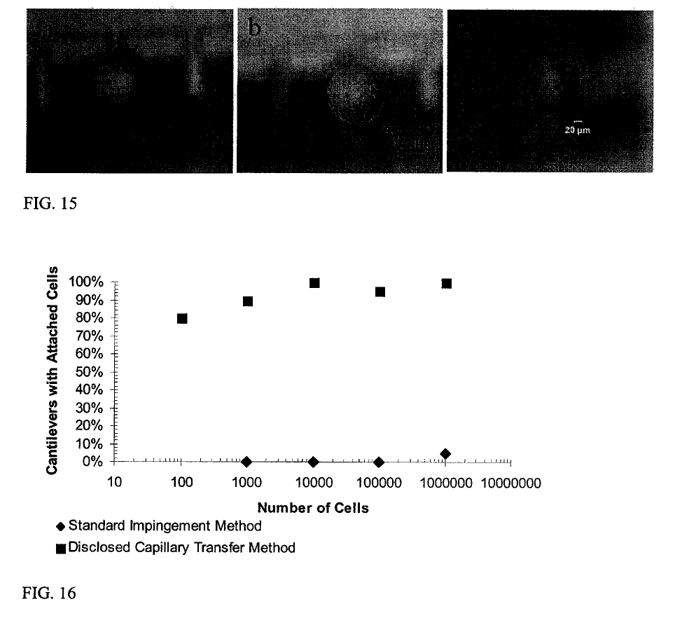

FIG. 15. presents optical micrographs of (a) a colloidal probe prior to cell seeding, (b) the same colloidal probe illustrated in (a) after lateral translation induced cell seeding (shown in FIG. 10.) using a 1000 MET-5A human mesothelial cells in growth media, (c) a similar colloidal probe as given in (a) and (b) seeded by electric potential induced capillary transfer under identical cell loading conditions (shown in FIG. 14). All images are of the same scale.

FIG. 16. is a graph illustrating the differential success rate between the standard and the disclosed cell attachment method for MET-5A human mesothelial cells on polystyrene microsphere-terminated cantilevers. For each cell concentration and method, twenty attempts were made. The results indicate the percentage of cantilevers with at least one attached cell out of twenty trials for each seeding technique and total cell concentration. Note that the standard impingement method refers to the method where cells are injected towards a microparticle immersed in growth media.

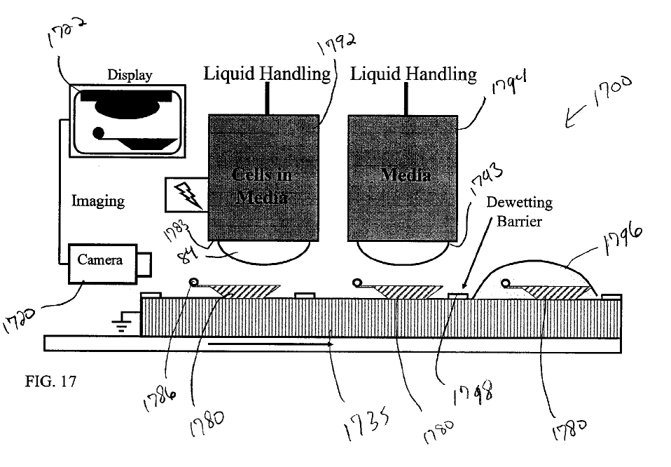

FIG. 17 shows a schematic of an automated device 1700 for cell seeding based on capillary wetting. A similar manual device was used in FIGS. 12, and 14. A translatable platform 1735 has positioned thereon a series of cantilevers 1780 with particles 1786 associated on the free end of the cantilever. A first media dispenser 1792 contains media with cells and creates a droplet of media 84 via an aperture 1783 defined on the bottom of the dispenser 1792. A second media dispenser 1794 contains media without cells and dispenses an amount of media 1796, via an aperture 1793 defined in the bottom thereof, to encompass the cantilever 1780. The dewetting barrier 1798 is provided to contain media between cantilevers. The platform 1735 moves the cantilevers 1780 for placement under the dispensers. The device also includes a camera 1720 that is positioned and configured so as to capture the seeding and/or media encompassing process. The cameral 1720 is connected to a display unit 1722.

### Single Adherent Cells

In certain situations it is desired to study the adhesion between single adherent cells, particularly if results are to be compared with single detached cells (lipid anchored) or confluent cell layers. The surface expression of cells in these three physiologically relevant states can be considerably different. By using dilute seeding concentrations, single adherent cells can also be attached to the end of a sphere following the methods describe above. However, alternatively selective hydrophobization of the cantilever can be performed to induce capillary confinement of the wetting drop to the end of the cantilever itself.

### An Integrated Device for the Application of Living Cell Force Sensors

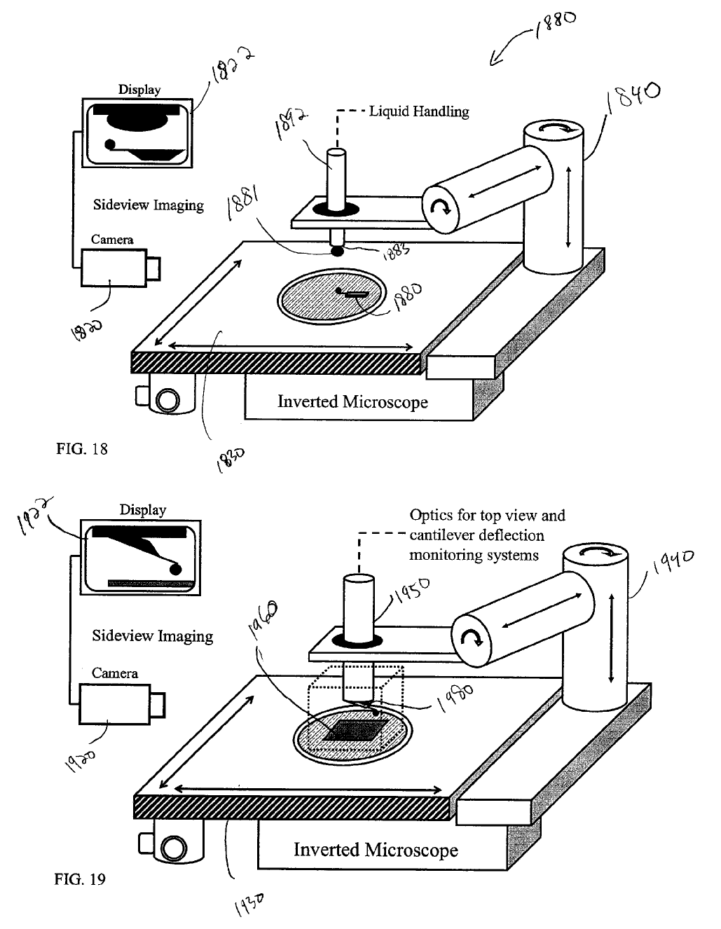

In order to extend the use of living cell force sensors to laboratories that do not have AFM/SPM facilities the inventors have developed sensor designs and suitable equipment integrated equipment for both the seeding and utilization of these sensors under any laboratory setting. Standard AFM/SPM cantilevers are manufactured from silicon or silicon nitride and have selected dimensions that impede normal thermal vibrations greater than 1 to 2 nm in amplitude. The primary reason for this is that for conventional AFM's this amount of deflection amplitude is considered large and contributes to the overall noise of the system. Moreover, in typical AFM force measurements, separation distances of 1-2 nm can illustrate a large difference in the measured force. However, the forces measured between living cells and surfaces normally operate over several microns. Hence, the noise levels of the cantilevers can therefore be comparable to ˜1 micron, which essentially means that softer and longer cantilevers can be fabricated and applied for interaction measurements outside of standard AFM/SPM equipment which necessitate picometer tolerances. In general, this means that cantilever systems can be fabricated and used to contain cells that are by standard definition unsuitable for AFM/SPM use but are suitable for use under standard optical microscopy. This will allow for less tolerance in cantilever manufacture and the use of new materials such as polymer and plastic films that could considerably reduce fabrication costs. It is believed that optical means such as interferometery, diffraction, image blurring and side view cantilever imaging, can optionally be coupled with imaging software to provide suitable interaction force interpretation for a wide range of cell adhesion studies. Furthermore, other methodologies of sensing deflection of the cantilever include, but are not limited to, capacitance and resistance. Thus, such more facile means of sensing an interaction between the cantilever avoids the need to purchase and/or use expensive afm/spm machines. Moreover, it should be noted that the use of a large microparticle at the end of the cantilever facilitates enhanced cell-surface contact area, which in turn leads to stronger binding in the presence of a ligand-receptor pair. Hence, some traditional AFM cantilevers with low spring constants could also be used for simple optical detection. In addition, capacitance based cantilevers could also be easily incorporated for use. A general schematic of a suitable device is given in FIGS. 18-20. For XYZ translations, inexpensive piezos or stepping motors may be used. Such devices would cost only a small fraction of a standard AFM and could be integrated to work with existing devices such as standard inverted microscopes.

FIG. 18 shows a schematic of an integrated device 1800 for fabricating living cell-terminated microcantilevers and measuring interaction forces between said cantilever and test surfaces is presented. In this view, a mode suitable for capillary transfer of living cells onto cantilevers is presented. The device comprises a translatable platform 1830 onto which a cantilever 1880 is positioned. A liquid media dispenser 1892 is provided that is associated with an adjustable mechanism 1840. The dispenser 1892 creates a droplet 1881 out an aperture 1883 and the droplet 1881 is brought into contact with the cantilever 1880 either by movement of the mechanism 1840 or by movement of the platform 1830. The device 1800 also includes a camera 1820 and display unit 1822 for visualizing the interaction of the droplet 1881 with the cantilever 1880.

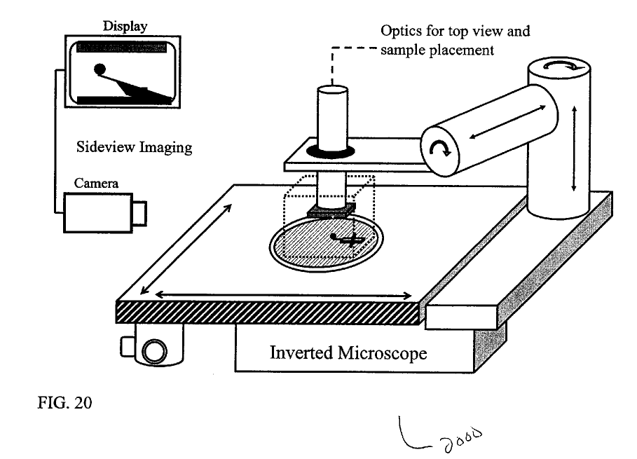

FIG. 19 shows a schematic of an integrated device 1900 for fabricating living cell-terminated microcantilevers and measuring interaction forces between said cantilever and test surfaces is presented. In this view, a mode suitable for measuring interaction forces between cell probes and test substrates is presented. A cell seeded cantilever 1980 is attached to a monitoring device 1950 that is associated with an adjustable mechanism 1940. A testable sample 1960 is positioned on a translatable platform 1930. The cantilever 1880 is lowered by the adjustable mechanism 1940 to be brought in proximity or contact with the surface of the sample 1960 so that interactive forces between the sample 1960 and cantilever 1980 can be observed. The device 1900 also includes a camera 1920 and 1922 for additional visual display of the interaction between the cantilever 1980 and sample 1960. FIG. 20 shows an alternative arrangement to that shown in FIG. 19. The device 2000 shown in FIG. 20 is similar to that shown in FIG. 19 except that the sample is provided on the monitoring device and the cantilever is provided on the platform.

### Diagnostic Kits

Current disposable kits for the detection of disease and/or other ailments typically rely on soluble factors such as the presence of specific proteins or other moieties in solution (e.g., urine, blood, saliva, etc.) for detection. The presence of molecules on cell surfaces has the potential to provide an alternative strategy for the diagnosis and/or early diagnosis of disease. However, conventional measures for identifying cell surface antigens involve tedium and a well-qualified trained user for analysis. In many cases, detection involves the use of expensive analytes such as calorimetric or fluorescent labels that is used to stain cells, which are then subsequently inspected for the presence or absence of said labels. These techniques often involve the use of multiple processing steps and require equipment for microscopic observation, hence are not readily accessible as a bedside diagnostic. As well, standard cell adhesion assays require numerous cells and a similar tedium that is also not amenable to bed side diagnostic formats. In contrast, our cantilever based technology requires a very small number of cells and the seeding process effectively extracts the cells from biological media-avoiding issues associated with interference molecules. The inventors have conceived general strategies in which living cell force sensors can be used in simple bed side diagnostics formats.

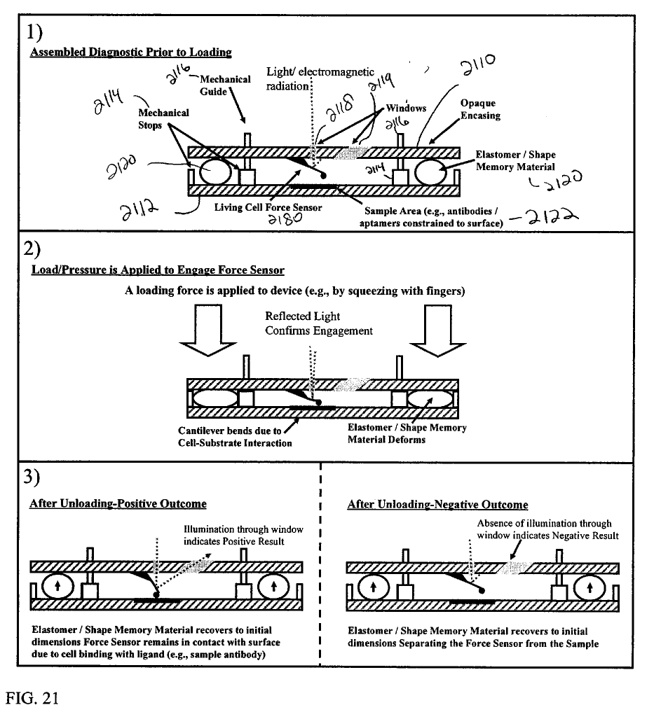

In one embodiment, schematically presented in FIG. 21. a force sensor is integrated into a simple device that is composed of an upper part 2110 (e.g. plate) and lower part (e.g. plate) 2112. A cantilever 2180 is secured to a underside of the upper part 2110. Secured subjacent to the cantilever 2180 but on the topside of the lower part 2112 is a sample 2122. In alternative embodiments, the arrangement between the cantilever 2180 and sample 2122 is switched. Disposed between the upper and lower parts 2110 and 2112 is a shape memory component 2120. Mechanical guides 2116 and mechanical stops 2114 are associated with the encasement formed by the upper and lower parts 2110, 2112. The upper part 2110 and lower part 2112 are pressed together bringing the cantilever 2180 in proximity to or contact with the sample 2122. The mechanical guides 2116 direct the alignment of the two parts 2110, 2112. The mechanical stops 2114 govern the degree to which the upper and lower parts 2110, 2112 are brought together. The shape memory component 2120 causes the upper and lower parts 2110, 2112 to separate after the depression is released. Light is directed through window 2118 defined in the upper part 2110. Upon the upper and lower parts 2110, 2112 being pressed together and released, a positive outcome (interactive force between cantilever 2180 and sample 2122) can be determined by whether light reflects and is directed out of window 2119. This is due to the cantilever 2180 being in a deflected position as a result of the interaction with the sample 2122. A negative result is determined if no light is directed out of the window 2119 upon release of the upper and lower parts 2110, 2112. indicated in section 1, then pressed together as indicated in section 2, and a positive or negative result is determined by the final cantilever position as indicated in section 3. The use of a simple polydimethyl siloxane elastomer or the like, may be used as the shape memory component 2120, to provide an automatic restoring force which will serve to slowly increase the distance from the upper and the lower part in order to determine whether or not cell adhesion has occurred. In the scenario present, the detection of reflected light by the cantilever is used for the interpretation of a positive or negative result. Alternatively, other methods could be use such as light obstruction, holography, capacitance based electrical signaling etc.

### Datamining Applications

Because the living cell force sensors disclosed here are of micron-scale dimensions and can be positioned to interact with spatially defined areas, once automated, they could be used to datamine cell surfaces for the discover of new targeting ligands. Because of the small number of cells that can be placed at the end of a probe this technique could be combined with lab on chip methods for identifying the corresponding cellular gene expression that results in said ligands being expressed on the surface of the cells of interest.

More importantly since this technique can be used to detect single molecule binding it could be used in combination with separation protocols and mass spectroscopy to identify new ligands on the cell surface. An example of an application is as follows:

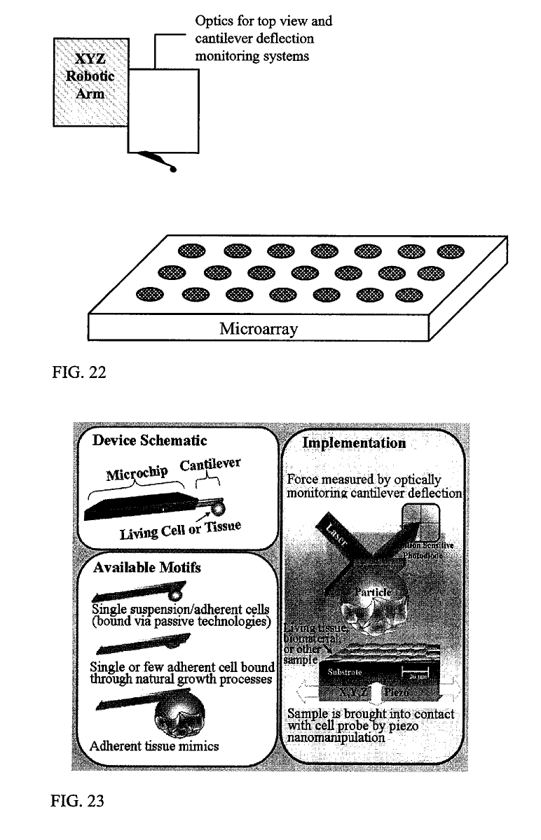

Suppose a new targeting molecule for a cell with a specific gene expression was desired. One could simply prepare a cantilever with said test cell, and scan a micro array of potential ligands that are surface-constrained (e.g., by covalent coupling). Positive spots are identified. The starting materials for said positive spots are refined through separation methods such as liquid chromatography to spot a new plate, which is then read by said force sensor. Likewise the positive spots are indicated and refined or sent for mass spectroscopy and other analytical techniques to determine their chemical makeup. Following such protocols could be used to find new ligands for cell surfaces without the need of apriori knowledge of the receptor. This is extremely important since cell surface proteins, etc. are very difficult to analyze and many of which are still not known. By screening using living cell force sensors devices such as that roughly depicted in FIG. 22. could be used to data mine for potentially new cell surface ligands. Also, negative selection and comparative methods can be incorporated to identify key binding difference between viable cells in the diseased and/or healthy state. Such a format could be also used with microarrays of living cells, for instance to attempt to determine where a cancer cell is likely to metastasize to, and many more important applications.

The subject application relates to pending PCT/US06/10828; filed Mar. 23, 2006. The teachings of the '828 application are incorporated herein to the extent they are not inconsistent with the teachings herein. The '828 application discusses several methods of detecting force interactions between a probe and a candidate structure or other sample. Those skilled in the art will appreciate that the embodiments described herein could be implemented in a similar fashion.

While the principles of the invention have been made clear in illustrative embodiments, there will be immediately apparent to those skilled in the art, in view of the teachings herein, many modifications of structure, arrangement, proportions, the elements, materials, and components used in the practice of the invention, and otherwise, which are particularly adapted to specific environments and operative requirements without departing from those principles. The appended claims are intended to cover and embrace any and all such modifications, with the limits only of the true purview, spirit and scope of the invention.

The references referred to herein are incorporated herein in their entirety to the extent they are not inconsistent with the teachings herein.

What is claimed is:

1. A living cell force sensor comprising a cantilever unit having a lever portion and a probe portion provided at a free end of said lever portion, said probe portion comprising a hydrophobic layer and one or more living cells constrained to said probe portion via at least partial association with said hydrophobic layer.

2. The sensor of claim 1, wherein said probe portion comprises an attachment surface; a plurality of hydrophillic spacer molecules attached to said attachment surface at one end; and a plurality of hydrophobes attached to said plurality of hydrophilic spacer molecules thereby forming said hydrophobic layer.

3. The sensor of claim 2, wherein said plurality of hydrophilic spacer molecules comprises PEG.

4. The sensor of claim 2, wherein plurality of said hydrophobes comprises oleyl moieties.

5. A living cell force sensor comprising a cantilever unit having a lever portion and a probe portion provided at a free end of said lever portion, said probe portion comprising a hydrophobic layer and one or more emulsion droplets or liposomes constrained to said probe portion via at least partial association with said hydrophobic layer.

6. A method of screening for biologically active molecules or nanostructures comprising:
providing a plurality of molecule candidates or nanostructure candidates on a substrate; and
interacting said candidates with the living cell force sensor of claim 1;
wherein a candidate exhibiting adhesion to said living cell force sensor is identified as biologically active.

7. A method of producing a cantilever having a lever portion and a probe portion, wherein cells are seeded on said probe portion comprising:
generating a droplet of media containing a suspension of cells, wherein said droplet is held by a dispenser;
moving said dispenser or said cantilever, or both, so as to bring said droplet in proximity or contact with said probe portion; and
displacing said dispenser or said cantilever, or both, so as distance said droplet away from said probe portion, whereby cells in said droplet become associated with said probe portion.

8. The method of claim 7, wherein said moving and displacing steps effectuate cell seeding by drop advancement and retraction.

9. The method of claim 7, wherein said moving and displacing steps effectuate cell seeding by normal translation.

10. The method of claim 7, wherein said moving and displacing steps effectuate cell seeding by lateral translation.

11. The method of claim 7, wherein said moving and displacing steps effectuate cell seeding by applied electric potential.

12. A method for seeding cells onto a cantilever comprising positioning two or more cantilevers onto a movable platform, said cantilevers each having a probe portion;
forming a droplet via a dispenser comprising a receptacle for holding media containing cells, said dispenser having an aperture through which an amount of media is dispensed to form said droplet;
laterally moving said two or more cantilevers so as to bring a communicative portion of at least one of said two or more cantilevers in proximity with or contact with said droplet; and
laterally transporting said two or more cantilevers so as to displace said communicative portion away from said droplet, whereby cells from said droplet are associated said communicative portion to achieve a cell-seeded cantilever.

13. The method of claim 12, further comprising subjecting said cell-seeded cantilever to an amount of media sufficient to encompass said cell-seeded cantilever.

14. A system for producing a cell seeded cantilever comprising
a dispenser comprising a receptacle for holding cell containing media, said dispenser comprising an aperture defined on at least one end adapted for dispensing a droplet of media; and
a platform for holding a cantilever;
wherein said dispenser is mechanically adjustable in an X, Y and/or Z axis; or
wherein said platform is mechanically adjustable in an X, Y, and/or Z axis, or wherein both dispenser and platform are mechanically adjustable.

15. The system of claim 14, wherein said platform is static and said dispenser is adjustable.

16. The system of claim 14, wherein said platform is adjustable and said dispenser is static.

17. The system of claim 14, wherein said dispenser is attached to an adjustable mechanism having at least 1, 2, 3, or 4 degrees of freedom.

18. The system of claim 14, further comprising a camera positioned so as to capture communication between said droplet and said cantilever.

19. The system of claim 18, further comprising a display unit connected to said camera.

20. The method of claim 7, wherein said probe portion comprises a carrier particle.

21. The method of claim 12, wherein said probe portion comprises a carrier particle.

22. A diagnostic kit comprising a first part having a topside and underside surface and a second part having a topside and underside surface, said first part and second part movably enagaged to each other; a cantilever comprising a lever portion and probe portion, said cantilever secured to said underside surface of said first part; a sample disposed on said topside surface of said second part, said cantilever and said sample being positioned on said first and second part, respectively such that when a force is applied to urge said first part and second part toward each other, said probe portion is brought into proximity with or contact with said sample.

23. The diagnostic kit of claim 22, further comprising a shape memory component that displaces said first part from said second part to a predetermined position after release of said force.

24. The diagnostic kit of claim 23, wherein predetermined position is generally at the position of said first and second parts prior to said force being applied.

25. The diagnostic kit of claim 22, wherein said first part comprises a first window and a second window.

26. The diagnostic kit of claim 25, wherein said first and second windows are configured such that light is directed through said first window and reflected off said cantilever and directed through said second window following release of said force if said probe portion interacts with said sample.
